(19) United States
(12) Patent Application Publication
(10) Pub. No.: US 2007/0116730 A1
(43) Pub. Date: May 24, 2007

(54) Pharmaceutical compositions

(75) Inventors: Robert D. Simmons, Martinsville, NJ (US); Allan J. Weingarten, Westfield, NJ (US); Yuping Li, Bridgewater, NJ (US); Edward Whittem, E. Maitland (AU)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(73) Assignee: Schering-Plough Animal Health Corp.

(21) Appl. No.: 11/600,418

(22) Filed: Nov. 16, 2006

Related U.S. Application Data
(60) Provisional application No. 60/738,524, filed on Nov. 21, 2005.

(51) Int. Cl. A61K 31/60; A61K 31/485; A61K 31/192; A61K 9/00

(52) U.S. Cl. 424/400; 514/282; 514/569; 514/570; 514/165

(57) ABSTRACT

A method of providing systemic analgesia to cats, dogs and other small mammals by the ophthalmic administration of opioids is disclosed. Compositions for use in such a method are also disclosed.

### CROSS REFERENCE TO RELATED APPLICATIONS

This application is a non-provisional application that claims the benefit of priority under 35 U.S.C. § 119(e) of provisional application U.S. Ser. No. 60/738,524 filed Nov. 21, 2005, the contents of which is hereby incorporated by reference in its entirety.

### FIELD OF THE INVENTION

The present invention relates to compositions and methods for providing systemic analgesia, and more particularly to the ophthalmic administration of opioid analgesics to cats, dogs and other mammals.

### BACKGROUND OF THE INVENTION

All patents, applications, publications, test methods, and other materials cited herein are incorporated by reference.

Pain activates the stress hormone systems of the body and contributes to morbidity and mortality. Relief of pain (analgesia) in animals can safely be provided by opioids titrated to effect. Opioids can provide profound analgesia with minimal cardiovascular side effects, are safe alone and in combination with anesthetics, and are reversible if an adverse event should occur.

Historically, pharmacologic agents, including opioids, have been administered through systemic injection (subcutaneous, intramuscular or intravenous), epidurally, intrathecally (into the subarachnoid space), sublingually, orally, rectally and transdermally to provide analgesia. With the exception of epidural and intrathecal delivery, administration of these agents provides systemic drug delivery to produce analgesic effects. Epidural and intrathecal administration involves the direct administration of an analgesic agent to receptors in the spinal cord involved in spinal transmission of pain (e.g. opioid receptors), bypassing the need for systemic exposure to the pharmacologic agent in question.

Opioids produce analgesia by binding with opioid receptors within the nervous system to block the transmission of the pain impulse to the higher brain centers, thus diminishing or blocking the perception of pain. There are three types of well-characterized opioid receptors: mu, kappa and delta. Most of the clinically useful opioid medications are mu agonists.

TORBUGESIC-SA (butorphanol tartrate) is a veterinary product approved in the U.S. for perioperative analgesia. Butorphanol is an opioid agonist/antagonist.

Full opioid agonists such as oxymorphone, morphine, meperidine and fentanyl can provide profound analgesia to animals and are safe for use in combination with anaesthetics. For example, hydromorphone is used in veterinary practice as a perioperative analgesic by the injectable route of administration. However, parenteral administration is not practical for use by animal owners without veterinary training. Oral formulations of many opioids are also available, but opioid agonists have a low systemic bioavailability when administered orally due to extensive hepatic first-pass metabolism. Fentanyl has been administered transdermally via adhesive drug-filled patches, but such patches are expensive and inconvenient to use on fur-covered animals. Moreover, transdermal patches require up to six hours to achieve a therapeutic effect, and analgesia must be provided by other means in the interim.

In addition to the shortcomings of present methods for the administration of opioids to animals discussed above, the possibility of overdose and the potential for abuse by humans has limited their use in animals.

U.S. Pat. No. 5,589,480 relates to a method for inducing analgesia in inflamed skin by topically administering to the skin an opioid analgesic agent in an amount that is ineffective for induction of systemic analgesia. According to this patent, effective analgesia must be induced in the “substantial absence of transdermal delivery of the opioid analgesic agent to the systemic circulation.”

U.S. Pat. No. 6,011,022 relates to a method of inducing analgesia in skin or mucosal tissue, comprising ocularly administering an analgesic agent that affects peripheral muscarinic receptors, which amount is systemically ineffective for induction of analgesia, and whereby the analgesia of the skin or mucosal tissue is induced in the substantial absence of transdermal or transmucosal delivery of the analgesic agent to the central nervous system. While oxymorphone and morphine are mentioned as analgesic agents that may be used in conjunction with a muscarinic receptor agonist analgesic, they are not themselves muscarinic receptor agonists. “Mucosal tissue” is specifically defined in the specification as excluding the conjunctiva of the eye.

The administration of certain veterinary drugs by the ophthalmic route is known, but not for the provision of systemic analgesia. For example, U.S. Pat. No. 5,543,434 relates to the nasal or ocular administration of ketamine to control chronic pain. U.S. Pat. No. 6,191,126 B1 is directed to the administration of kappa opioid agonists to the eye to treat ocular pain. This patent stresses that kappa opioids act on receptors in peripheral tissue, while mu opioids relieve pain by activating receptors in the brain. The local action of kappa opioids is said to be an advantage over systemic action. Accordingly, the present invention is only suitable for treatment of pain in the ophthalmic tissues, not systemic analgesia.

In view of the foregoing limitations and shortcomings of the prior art formulations and methods, as well as other disadvantages not specifically mentioned above, it is apparent that there still exists a need in the art for improved means for systemically inducing analgesia.

### SUMMARY OF THE INVENTION

Accordingly, there are disclosed pharmaceutically acceptable compositions for ophthalmic administration to an animal and methods for the use thereof. Such compositions comprise buprenorphine and a pharmaceutically acceptable carrier system comprising a solvent consisting of a water phase and/or organic phase and, optionally, at least one penetration enhancing agent and/or a stabilizing or tonicity adjustment agent. The present composition can also optionally include a non-opioid analgesic, such as a non-steroidal anti-inflammatory drug (NSAID), N-methyl-d-aspartate (NMDA) receptor antagonist, alpha-2 adrenergic receptor agonist, or a sodium channel blocker.

With the foregoing and other objects, advantages and features of the invention that will become hereinafter apparent, the nature of the invention may be more clearly understood by reference to the following detailed description of the invention and the appended claims.

### BRIEF DESCRIPTION OF THE FIGURE

FIG. 1 is a graph showing the mean plasma concentration of buprenorphine versus time in six healthy cats following ophthalmic administration of a buprenorphine formulation at a dose of either 0.025-0.05 mg/kg or 0.05-0.10 mg/kg. Data for cats dosed at 0.025-0.05 mg/kg are represented by the solid line and have −1 SD shown for each timepoint; data for cats dosed at 0.05-0.10 mg/kg are represented by the solid line and have +1 SD shown for each timepoint.

### DETAILED DESCRIPTION OF THE INVENTION

It has been found that effective concentrations of opioids in the systemic circulation for the purpose of providing systemic analgesia can be achieved by the ophthalmic route of administration. By using the ophthalmic route of administration, liver/gut wall (“first-pass”) metabolism of the opioid is avoided, which may enhance bioavailability relative to oral dosing.

The present invention relates to an opioid analgesic product for providing systemic analgesia, e.g., pre-emptive and perioperative analgesia, for mammals such as cats and dogs. The present invention comprises at least one opioid analgesic in a pharmaceutically acceptable vehicle. The compositions of the present invention can be used to simultaneously prevent or reduce the pain associated with surgery or injury. Use for the treatment of chronic pain associated with, e.g., neoplasia, osteoarthritis, pruritis, etc. is also contemplated.

As used herein, the term “opiate” means any preparation or derivative of opium. The term “opioid” refers to both opiates and synthetic or semi-synthetic narcotics resembling opiates.

As used herein, the term “water phase” means a solvent system comprised of water, isotonic solution, a buffer system and/or any solvent mixable with water.

As used herein, the term “organic phase” means a solvent system comprised of any organic solvent or solvent system mixable or not mixable with water.

Active ingredients include opioid analgesics, in particular those having agonist activity at the mu opiate receptor, such as buprenorphine, morphine, diamorphine, meperidine, methadone, etorphine, levorphanol, fentanyl, alfentanil, sufentanil, oxycodone, hydrocodone, codeine, and oxymorphone. Particularly preferred is buprenorphine because of a wider safety margin and longer duration of activity.

In the preferred embodiment, the formulation is long acting, e.g. it is administered up to three times a day as needed. Because it is a long acting formulation, as opposed to a short acting formulation, one particular advantage of the present invention is the reduced dosing frequency and offering convenience for the person administering the product.

It will also be appreciated that the present invention encompasses, in one aspect, methods of alleviating pain by administering, for example, a pharmaceutically acceptable composition comprising, for example, buprenorphine, to an animal by ophthalmic administration. Dosing administration may also be accomplished, for instance, by applying multiple or single drops to the eye of the animal.

Plasma concentrations of buprenorphine, following single dose ophthalmic administration at a dosing range of about 0.005 to about 0.1 mg/kg achieve a Cmax of about 5 to about 60 ng/mL at a Tmax of about 0.25 hours.

Metabolites of opioid analgesics that have analgesic activity may also be used. Such metabolites include, e.g., analgesically active glucuronide and sulphate forms of opioids such as morphine-6-glucoronide.

Due to possible problems created by the unpleasant odor of the drug, low bioavailability or the potential for local analgesic effect, it may be desirable to use a prodrug form of such opioid. Particularly preferred prodrug forms are those in which the 3-phenolic hydroxy group is esterified. Examples of prodrug derivatives suitable for use in the present invention include those disclosed in U.S. Pat. Nos. 4,668,685 and 4,673,679, both assigned to DuPont.

In another embodiment, the present invention allows for the inclusion of a non-opioid analgesic, such as an NSAID. Preferred NSAIDs, include acemetacin, acetylsalicylic acid (aspirin), alminoprofen, benoxaprofen, bucloxic acid, carprofen, celecoxib, clidanac, deracoxib, diclofenac, diflunisal, dipyrone, etodolac, fenoprofen, fentiazac, firocoxib, flobufen, flufenamic acid, flufenisal, flunixin, fluprofen, flurbiprofen, ibuprofen, indomethacin, indoprofen, isoxicam, ketoprofen, ketorolac, meclofenamic acid, mefenamic acid, meloxicam, miroprofen, nabumetone, naproxen, niflumic acid, oxaprozin, oxepinac, phenylbutazone, piroxicam, pirprofen, pramoprofen, sudoxicam, sulindac, suprofen, tepoxalin, tiaprofenic acid, tiopinac, tolfenamic acid, tolmetin, trioxaprofen, zidometacin, or zomepirac, pharmaceutically acceptable salts thereof and mixtures thereof. Particularly preferred NSAIDs include carprofen, deracoxib, etodolac, firocoxib, flunixin, ketoprofen, meloxicam and tepoxalin. Preferred NMDA receptor antagonists include memantine, ketamine, tiletamine, and pharmaceutically acceptable salts thereof and mixtures thereof. A particularly preferred NMDA receptor antagonist is ketamine. Preferred alpha-2 adrenergic receptor agonists include clonidine, detomidine, dexmedetomidine, fadolmidine, medetomidine, moxonidine, romifidine, xylazine, and pharmaceutically acceptable salts thereof and mixtures thereof. Particularly preferred alpha-2 adrenergic receptor agonists include detomidine and xylazine. Preferred sodium channel blockers include benzocaine, bupivacaine, lamotrigine, levobupivicaine, lidocaine, lignocaine, oxybuprocaine, prilocaine, proxymetacaine, ropivicaine, and pharmaceutically acceptable salts thereof and mixtures thereof. Particularly preferred sodium channel blockers include bupivacaine and lidocaine.

In general the formulations of the present invention will contain from about 0.01 to about 10% of the opioid(s) in an ophthalmically acceptable vehicle. Preferably, from about 0.01 to about 1% of the opioid(s) in an ophthalmically acceptable vehicle. The amount of the opioid(s) may be varied to alter the dose volume and/or the dosage schedule. The quantity of a second analgesic such as an NSAID will depend on compatibility with ocular tissues, synergy with the opioid and bioavailability and will be titrated to effect.

The compositions of the present invention may take various forms. For example, they may be a gel, liquid, or ointment.

The solvent used in the composition may contain water, tonicity adjusting agents and/or other solvents. Suitable solvents include water, sterile isotonic solution, glyceryl formal, dimethylformamide, N-methyl-pyrrolidone, 2-pyrrolidone, glycol, propylene glycol, polyethylene glycol, diethylisosorbide, ethanol, isopropanol, 1,2-propanediol, glycerin, triethyl citrate, benzyl alcohol, dimethylisosorbide, C₂-C₉ alkylene diols, e.g., butylene diol, pentylene glycol, neopentyl diol, propylene glycol diethylene glycol, monoethyl ether or like compounds such as di C₂-C₅ alkylene diol, mono C₁-C₄ alkyl ethers, e.g., dipropylene glycol, mono propyl ether, mono propyl ether, and mono ethyl ether. Preferred solvents include 2-pyrrolidone, glyceryl formal, dimethylformamide, N-methyl-pyrrolidone, propylene glycol, polyethylene glycol, diethylisosorbide, ethanol, isopropanol, 1,2-propanediol, glycerin, triethyl citrate, benzyl alcohol, dimethylisosorbide, glycol, water and sterile isotonic solution. Particularly preferred solvents include water, sterile isotonic solution, ethanol and propylene glycol. Preferably, such a solvent is present in an amount of up to about 97.5% by weight of the formulation.

Suitable tonicity adjustment agents may include, but are not limited to, sodium and potassium chloride, glucose, sorbitol, polyhydric alcohols such as glycerol, polyethylene glycol and propylene glycol and polyalcohols such as mannitol. Preferred tonicity adjustment agents include sodium chloride, propylene glycol and polyalcohol. The tonicity adjustment agents may be employed in an amount effective to adjust the osmotic value of the final composition to a desired value, typically from about 250 to about 350 mOsmols/kg in order to approximate the osmotic pressure of normal lachrymal fluids which is equivalent to a 0.9 percent solution of sodium chloride.

Suitable penetration enhancers may include both lipophilic and hydrophilic components, taking into consideration their ocular tolerance. Suitable penetration enhancers include, but are not limited to, an alcohol, a nonionic solubilizer or an emulsifier. Suitable penetration enhancers include, but are not limited to, water, ethylene glycol, propylene glycol, dimethyl sulfoxide (DMSO), dimethyl polysiloxane (DMPX), oleic acid, caprylic acid, isopropyl alcohol, 1-octanol, ethanol (denatured or anhydrous), and other pharmaceutical grade or absolute alcohols with the exception of methanol. Other penetration enhancers include, sulphoxides and similar chemicals, such as DMSO, Decamethonium Br, Tween20, Brj 35, EDTA, glycocholate Na, sodium salt of hyaluric acid, hydroxy propyl cyclodextrin (and other cyclodextrins compatible with ocular tolerance), dimethylacetamide (DMA), dimethylformamide (DMF), etc., azone and related compounds, pyrrolidones, such as N-methyl-pyrrolidone (NMP), 2-pyrrolidone (2-pyrrol), etc., fatty alcohols, fatty acids and related structures, such as oleyl alcohol, oleic acid, linoleic acid, isopropyl myristate, etc., alcohols and glycols, such as ethanol, propylene glycol, lauryl alcohol esters and lauryl alcohol, etc., the esters of propylene glycol, such as propylene glycol monolaurate, surfactants, such as sodium lauryl sulphate (SLS), etc., urea, essential oils, terpenes and terpenoids, such as eucalyptus oil, 1,8-cineole, etc., phospholipids and solvents and related compounds, such as transcutol (ethoxydiglycol), etc. Preferred penetration enhancers are DMSO, Decamethonium Br, Tween20, Brj 35, EDTA, glycocholate Na, sodium salt of hyaluric acid, water, ethanol and hydroxypropyl cyclodextrin.

The viscosity of the vehicle may be increased or decreased as necessary by the use of various additional agents. The viscosity increasing agent may be a water-dispersible acid polymer, a polysaccharide gum, and/or a mixture thereof. Suitable agents for use in the compositions of the present invention include, but are not limited to, hydroxyethylcellulose, hydroxypropylmethylcellulose, polyvinyl alcohol, polyvinyl pyrrolidone, magnesium sulfate, propylene glycol, lanolin, glycerin, hydroxypropylcellulose, carboxyvinyl polymers known as “Carbopol” and other agents known to those skilled in the art to be suitable for use in the eye.

Emulsifiers suitable for use in the compositions of the present invention can be nonionic, anionic or cationic. Exemplary emulsifiers include, but are not limited to, polyethylene glycol (PEG) 30 dipolyhydroxystearate (e.g. ARLICEL P135, available from ICI Surfactants, Wilmington, Del.), PEG-40 stearate sorbitan oleate (e.g. CRILL 4, available from Croda, Inc., Parsippany, N.J.), polysorbate 80 (e.g. TWEEN 80, available from ICI Surfactants), fatty alcohols, lanolin and derivatives, polyethylene glycol stearate, fatty acid monoglycerides, sorbitan fatty acid esters, polyoxyethylene sorbitan fatty acid esters, lecithins and phospholipids.

One component of the organic solution is a solvent composed of compounds, such as suitable surfactants for the organic solution, which include, for example, monoglycerides or like compounds such as glyceryl mono-oleate, -laurate, -behenate, -eicosadioate, -sterate, or other fatty acid mono substituted glycerides.

Suitable film formers for the organic solution include, but are not limited to, polyacrylamide or other like compounds, which act as thickening agents such as other acrylamide copolymers, polyacrylate copolymers, cellulosic polymers and copoylmers, poly vinyl pyrrolidone polymers and copolymers, hydrocolloids, glycerol, propylene glycol and polyethylene glycol.

Other optional inert ingredients may be added to the present composition, as desired. Such ingredients include preservatives, chelating agents, antioxidants, stabilizers, tonicity adjustment agents, lubricants, humectants, emoilients, surfactants and wetting agents. Exemplary preservatives include, but are not limited to, benzalkonium chloride, benzethonium chloride, chlorobutanol, phenylmercuric acetate, phenylmercuric nitrate, thimerosal, methyl p-hydroxybenzoate (methylparaben) and propyl p-hydroxybenzoate (propylparaben). It will also be appreciated that the formulations of the present invention in another embodiment are self-preserving. Exemplary chelating agents include, but are not limited to, edetate sodium, citric acid, ethylenediamine tetraacetic acid (EDTA) and its salts, sorbitol, tartaric acid and phosphoric acid. Exemplary antioxidants include, but are not limited to, ethylenediaminetetraacetic acid, sodium bisulfite, sodium metabisulfite, thiourea, butylated hydroxyanisole, sodium monothioglycerol, ascorbic acid, cysteine hydrochloride, ascorbyl palmitate, butylated hydroxytoluene (BHT), lecithin, propyl gallate and alpha-tocopherol. Preferred stabilizers for use in the present invention include, but are not limited to, BHT or citric acid in a concentration of about 0.5% or less and monothioglycerol in a concentration of about 0.1% to 2% w/v. A particularly preferred stabilizer to prevent degradation of any of the active ingredients in the formulations of the present invention is BHT. Other suitable stabilizers include, but are not limited to, triethyl citrate, USP, NF, acetic acid, glacial acetic acid, fumaric acid, hydrochloric acid, diluted hydrochloric acid and malic acid. Exemplary humectants include, but are not limited to, glycerol, sorbitol, propylene glycol and glucose. Exemplary emoilients include, but are not limited to, hydrocarbon oils, waxes, wax esters (examples include mineral oil, petrolatum, microcystalline wax), vegetable and animal fats and oils, glycerol based esters, fatty acids, fatty alcohols, fatty alcohol ethers, lanolin and derivatives and phospholipids. Exemplary tonicity adjustment agents are defined above.

Preferably the pH of the compositions of the present invention is adjusted to maintain buprenorphine or buprenorphine HCI in solution. Preferably, the pH of the compositions of the present invention are between 3 and 10. An appropriate buffering agent may be added to maintain the pH. Suitable buffers include, but are not limited to, potassium chloride, sodium or potassium phosphates (monobasic and dibasic), sodium or potassium acetates, sodium or potassium borates (e.g., sodium tetraborate decahydrate), sodium or potassium citrates, sodium or potassium hydroxides and equivalents or mixtures thereof, and weak acids, such as acetic, boric, and phosphoric acids.

In order to prepare the composition of the present invention, the vehicle(s) or a portion of the vehicle(s), are added to the compounding vessel, followed by the remaining excipients and the actives. The mixture is mixed until all solids are dissolved or in suspension. Additional solvent(s) to bring the composition to final volume may be added if needed. Additives, such as those listed above, may also be included in the vessel and mixed into the formulation (the order of addition is not critical).

After application of the formulation, the opioid present in the composition is systemically absorbed. It is an advantage of the method of the present invention that it can provide a rapid initial absorption and remain detectable in plasma for at least 8 hours, thereby leading to a rapid onset and long duration of analgesic action. After administration, onset of analgesia begins within 30 minutes of application, and the duration of analgesic action generally lasts up to at least 8 hours.

An advantage of the present invention is that the invention could be formulated to exhibit a rapid absorptive phase and a prolonged plateau phase (slow absorptive phase). Other advantages of the present invention are the fact that animals in pain and/or animals on an opiate can be aggressive. Therefore, administration of the present invention has the advantage that an animal handler never has to go near the mouth/teeth of the animal, i.e., increased animal handler safety.

The method of the present invention, and the formulations to carry out the method, have other advantages over existing products, such as ease of administration for both the veterinary staff and the owner of the animal, reduction in side effects, etc. In the case of an adverse event, the activity of the opioid is reversible by administration of opioid antagonists, e.g. naloxone.

It is believed that the route of administration may improve the bioavailability of many analgesic agents such as opioids that undergo hepatic first-pass metabolism and gastrointestinal degradation when administered orally. It is possible that the metabolism of such compounds may be favorably affected by the route of administration.

The appropriate dosage can be determined according to the weight of the animal. As will be appreciated by one of skill in the art, if renal or hepatic function is compromised, drug dosage may need to be decreased to account for decreased elimination.

The compositions of the present invention may be packaged in many forms. Preferably the formulation is packaged as single-dose, single-use units. Such single-dose packaging overcomes problems of bacterial contamination of multiple-dose ophthalmic preparations and minimizes the likelihood of accidental acute overdosing.

The following examples are given for the purpose of illustrating the present invention and should not be construed as limiting the scope or spirit of the invention.

### EXAMPLE 1

| Ingredient | Conc w/w |
|---|---|
| Buprenorphine HCl | 0.5% |
| (Free Base Equivalent) | (0.46%) |
| Propylene glycol | 1.96% |
| Purified Water | qs % |
| HCl 0.1N | for pH adjustment |

This Example may be prepared according to customary procedures known to one of skill in the art. In one specific embodiment the formulation can be prepared and stored in two separate systems: organic phase and water-phase. The two systems can be combined to obtain the final formulation.

### EXAMPLE 2

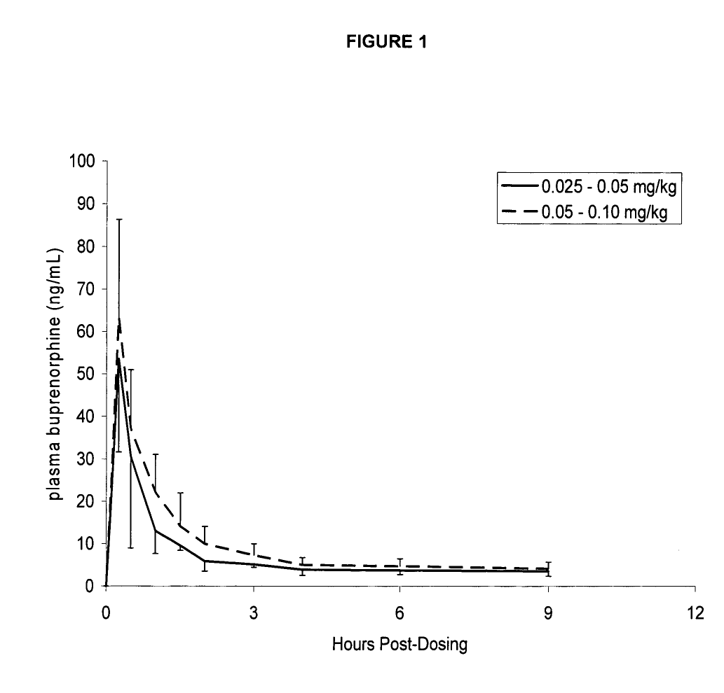

Six healthy cats were administered the formulation in Example 1 once using a dosage of 0.025-0.05 mg/kg, and then again using a dosage of 0.05-0.10 mg/kg. Following each dosing, serial blood samples were drawn at time 0 prior to dosing, then at 0.25, 0.5, 1, 1.5, 2, 3, 4, 6, and 9 hours after dosing. Plasma concentrations (ng/mL) of buprenorphine versus time were reported and graphically presented. The results are shown in FIG. 1.

These results display that the formulation described in Example 1 has a benefit, in that buprenorphine is detectable in plasma shortly after ophthalmic dosing, suggesting that analgesia will occur early. Secondly, plasma levels are detectable for at least 9 hours following dosing, suggesting that analgesia will be long-lasting.

Although certain presently preferred embodiments of the invention have been described herein, it will be apparent to those skilled in the art to which the invention pertains that variations and modifications of the described embodiment may be made without departing from the spirit and scope of the invention. Accordingly, it is intended that the invention be limited only to the extent required by the appended claims and the applicable rules of law.

We claim:

1. A pharmaceutically acceptable composition for ophthalmic administration to an animal comprising buprenorphine and a pharmaceutically acceptable carrier system comprising a solvent consisting of a water phase and/or an organic phase.

2. The pharmaceutically acceptable composition according to claim 1, further comprising a penetration enhancer.

3. The pharmaceutically acceptable composition according to claim 2, wherein the penetration enhancing agent is selected from the group consisting of DMSO, water, ethanol, Decamethonium Br, Tween20, Brj 35, EDTA, Glycocholate Na, sodium salt of hyaluric acid or hydroxypropyl cyclodextrin in an amount sufficient to enhance penetration of the buprenorphine.

4. The pharmaceutically acceptable composition according to claim 2, wherein the penetration enhancing agent is lipophilic and/or hydrophilic.

5. The pharmaceutically acceptable composition according to claim 1, wherein the solvent is selected from the group consisting of 2-pyrrolidone, glyceryl formal, dimethylformamide, N-methyl-pyrrolidone, propylene glycol, polyethylene glycol, diethylisosorbide, ethanol, isopropanol, 1,2-propanediol, glycerin, triethyl citrate, benzyl alcohol, dimethylisosorbide, glycol, water and sterile isotonic solution.

6. The pharmaceutically acceptable composition according to claim 5, wherein the solvent is water or isotonic sterile solution.

7. The pharmaceutically acceptable composition according to claim 1, further comprising a viscosity increasing agent.

8. The pharmaceutically acceptable composition according to claim 7, wherein the viscosity increasing agent is selected from the group consisting of a water-dispersible acid polymer, a polysaccharide gum, and/or a mixture thereof.

9. The pharmaceutically acceptable composition according to claim 1, wherein the composition has a pH in the range of about 3 to about 10.

10. The pharmaceutically acceptable composition according to claim 1, further comprising a tonicity adjustment agent.

11. The pharmaceutically acceptable composition according to claim 10, wherein the tonicity adjustment agent is selected from the group consisting of sodium chloride, propylene glycol and polyalcohol.

12. The pharmaceutically acceptable composition according to claim 11, wherein the polyalcohol is mannitol.

13. The pharmaceutically acceptable composition according to claim 11, wherein the tonicity adjustment agent is propylene glycol.

14. The pharmaceutically acceptable composition according to claim 1, further comprising a non-opioid analgesic.

15. The pharmaceutically acceptable composition according to claim 14, wherein the non-opioid analgesic is selected from the group consisting of acemetacin, acetylsalicylic acid (aspirin), alminoprofen, benoxaprofen, bucloxic acid, carprofen, celecoxib, clidanac, deracoxib, diclofenac, diflunisal, dipyrone, etodolac, fenoprofen, fentiazac, firocoxib, flobufen, flufenamic acid, flufenisal, flunixin, fluprofen, flurbiprofen, indoprofen, isoxicam, ketoprofen, ketorolac, meclofenamic acid, mefenamic acid, meloxicam, miroprofen, nabumetone, naproxen, niflumic acid, oxaprozin, oxepinac, phenylbutazone, piroxicam, pirprofen, pramoprofen, sudoxicam, sulindac, suprofen, tepoxalin, tiaprofenic acid, tiopinac, tolfenamic acid, tolmetin, trioxaprofen, zidometacin, zomepirac, and pharmaceutically acceptable salts thereof and mixtures thereof.

16. A method for inducing analgesia in an animal by ophthalmically administering buprenorphine in the pharmaceutically acceptable composition of claim 1.

17. A method for inducing a systemic analgesic effect in an animal by ophthalmically administering buprenorphine.

18. The method of claim 17, wherein the analgesic effect is for at least about 8 hours.

19. A method for inducing analgesia in an animal by ophthalmically administering buprenorphine, wherein at a dosing range of about 0.005 to about 0.1 mg/kg there is achieved a Cmax of about 5 to about 60 ng/mL at a Tmax of about 0.25 hours.
